(19) United States
(12) Patent Application Publication
(10) Pub. No.: US 2024/0399351 A1
(43) Pub. Date: Dec. 5, 2024

(54) UNSUPPORTED HYDROGENATION CATALYST, ITS PREPARATION AND APPLICATION THEREOF

(71) Applicants: CHINA PETROLEUM & CHEMICAL CORPORATION, Beijing (CN); SINOPEC RESEARCH INSTITUTE OF PETROLEUM PROCESSING CO., LTD., Beijing (CN)

(72) Inventors: Ting WANG, Beijing (CN); Huandi HOU, Beijing (CN); Ming DONG, Beijing (CN); Mengying TAO, Beijing (CN); Haiping SHEN, Beijing (CN); Jun LONG, Beijing (CN)

(21) Appl. No.: 18/698,574

(22) PCT Filed: Oct. 21, 2022
(86) PCT No.: PCT/CN2022/126654

§ 371 Date: Apr. 4, 2024

(30) Foreign Application Priority Data
Oct. 25, 2021 (CN) 202111244128.9
Oct. 25, 2021 (CN) 202111244521.8

(51) Int. Cl. B01J 31/22; B01J 37/04; C07C 5/10; C10G 45/50

(52) U.S. Cl. CPC B01J 31/2239; B01J 37/04; C07C 5/10; C10G 45/50; B01J 2231/646; B01J 2531/56; B01J 2531/64; B01J 2531/842; B01J 2531/845; B01J 2531/847

(57) ABSTRACT

Disclosed is an unsupported hydrogenation catalyst, its preparation and application thereof. The unsupported hydrogenation catalyst is composed of a complex formed by bonding a metal central atom or central ion with an organic ligand through coordination bond, wherein the metal is selected from the group consisting of Group VB metals, Group VIB metals, Group VIII metals, Group IB metals or combinations thereof that have a hydrogenation activity. The organic ligand comprises a hydrocarbyl moiety and a coordinating group, and forms a coordination bond with the metal central atom or central ion through an oxygen atom. The unsupported hydrogenation catalyst can be used for hydrogenation reaction of hydrocarbons, and has high oil solubility, dispersibility and hydrogenation activity.

### TECHNICAL FIELD

The present application relates to the field of hydrogenation catalysts, particularly to an unsupported hydrogenation catalyst its preparation and application thereof.

### BACKGROUND ART

At present, supported catalysts are hydrogenation catalysts for the processing of hydrocarbon compounds that have been studied for the longest time, and applied most widely in largest number of industrial applications. Supported catalysts are composed of catalytic active component, auxiliary catalytic active component and carrier, wherein the active component and the auxiliary catalytic active component are mainly metal, and the carrier is alumina, silica, kaolin, molecular sieve and other silica-alumina material or porous material. For example, in bimetallic catalytic reforming catalysts, Pt is used as an active component, Re or Sn is used as a promoter component, and Al₂O₃ is used as a carrier. Research shows that the carrier is an important component of the supported catalyst and is one of important factors influencing the performance of the catalyst. In the hydroprocessing of oil products, in order to meet the requirements of the process and the reaction on the fluidization property, the mechanical strength and the like of the catalyst, the carrier serves as a framework for the active component, so that catalysts with a shape, granularity and mechanical strength meeting the requirements of hydroprocessing is obtained; secondly, as the specific surface area of the active center is relatively small, and the carrier has a relatively large specific surface area, in order to improve the utilization rate of the active component, numerous researchers propose to uniformly disperse the active component on the surface of the carrier with large specific surface area, so that the surface area of the active component per unit mass can be significantly increased, and the function of the active component can be fully exerted; thirdly, the interaction between the carrier and the active component may influence the geometric configuration and catalytic activity of the active component and form a new active structure, thereby generating species overflow and the like; therefore, in recent years, research on the interaction between the metal component and the carrier has become a hot point in the development of catalysts for hydrogenation of distillate oils or heavy oils, which aims at improving the catalytic performance of the catalysts by modulating the influencing factors.

Hydrocarbon compounds or mixtures are present in the form of a liquid oily material under reaction conditions, while supported catalysts are usually solid, and thus hydrogenation reaction catalyzed by such catalysts belongs to heterogeneous catalytic reaction, which involves seven stages: namely, molecules of the feedstock diffuse to the outer surface of the catalyst→the molecules of the feedstock diffuse into the pore channel of the catalyst→the molecules of the feedstock are adsorbed onto the active center of the catalyst→the molecules of the feedstock undergo surface catalytic reaction with the active center of the catalyst→reaction products are desorbed from the surface of the active center of the catalyst→the reaction products diffuse outwards from the pore channel of the catalyst→the reaction products diffuse to a liquid phase system from the outer surface of the catalyst. Among those stages, the diffusion stage is an indispensable stage for the supported catalyst to catalyze the reaction of hydrocarbon compounds or mixtures, and influences the occurrence probability and efficiency of catalytic reaction. Therefore, many researchers focus on the modulation of carrier performance and the interaction between the carrier and the active metal, to improve the accessibility of the molecules of the feedstock to the active component of the catalyst, and develope hydrogenation catalysts that can meet the reaction requirements or improve the yield and selectivity of the target product.

International application publication No. WO200528106A1 discloses an alumina carrier having a large pore volume, a large specific surface area and a large number of mesopores, on which active metals Mo, W, Ni and Co are supported, thereby significantly promoting the cracking reaction of heavy hydrocarbons into light hydrocarbons.

Chinese patent application publication No. CN 101632938A discloses a hydrocracking catalyst obtained by using a high-silicon β molecular sieve efficiently synthesized by a plate-like micelle method and a modified Y-type molecular sieve as acidic component and carrier, which is modified and loaded with Group IA metal. When used in a heavy oil hydrocracking process, this catalyst shows higher hydrocracking activity and middle distillate selectivity.

Since the supported catalyst and the hydrocarbon feedstock belong to two different phases, the catalytic reaction always involves seven stages, which limits the reaction performance of the catalyst.

### DISCLOSURE OF THE INVENTION

An object of the present application is to provide an unsupported hydrogenation catalyst, its preparation and application thereof, the catalyst has high oil phase dispersity, and when used to catalyze the hydrogenation reaction of hydrocarbon compounds, the reaction belongs to a homogeneous catalysis reaction, thereby eliminating the diffusion stage of the heterogeneous catalysis reaction, which is beneficial to improving the hydrogenation activity.

To achieve the above object, in one aspect, the present application provides an unsupported hydrogenation catalyst, composed of a complex formed by bonding a metal central atom or central ion with an organic ligand through a coordination bond, wherein the metal is selected from the group consisting of Group VB metals, Group VIB metals, Group VIII metals, Group IB metals or combinations thereof that have a hydrogenation activity, the organic ligand comprises a hydrocarbyl moiety and a coordinating group that is —C(═O)—O group, and forms a coordination bond with the metal central atom or central ion through an oxygen atom, and the catalyst shows an infrared spectrum with characteristic peaks at positions of 700-1000 cm⁻¹, 1350-1450 cm⁻¹ and 1500-1610 cm⁻¹.

In another aspect, the present application provides a method for preparing an unsupported hydrogenation catalyst, comprising the steps of:
1) mixing a metal source or a dispersion thereof with an organic ligand compound;
2) reacting the mixture obtained in step 1) for 1-8 h at 100-350° C.; and
3) collecting the resulting liquid product,

wherein the metal source is selected from the group consisting of elemental metal, metal oxide, metal hydroxide, metallic oxyacid, metal inorganic salt or combinations thereof, and the metal in the metal source is selected from the group consisting of Group VB metals, Group VIB metals, Group VIII metals, Group IB metals, and combinations thereof that have a hydrogenation activity;

the organic ligand compound is selected from the group consisting of C4-C20 organic carboxylic acids or anhydrides thereof; and

the molar ratio of the organic ligand compound to the metal in the metal source is 1-10:1.

In yet another aspect, the present application provides a hydrogenation catalyst composition, comprising the unsupported hydrogenation catalyst according to the present application and at least one organic ligand compound and/or at least one organic solvent, wherein the organic ligand compound is selected from C4-C20 organic carboxylic acids, the organic solvent is selected from the group consisting of aliphatic hydrocarbons, aromatic hydrocarbons, alicyclic hydrocarbons, halogenated hydrocarbons, alcohol solvents, ether solvents, ester solvents, ketone solvents or combinations thereof.

In yet another aspect, there is provided the use of the unsupported hydrogenation catalyst or hydrogenation catalyst composition according to the present application in the hydrogenation of hydrocarbons.

In yet another aspect, the present application provides an unsupported catalyst composition, comprising, by weight, from 10% to 45% of a hydrogenation catalyst component, from 45% to 80% of a dispersing medium and from 1.0% to 10% of an activator, wherein:

the hydrogenation catalyst component is consisted of the unsupported hydrogenation catalyst according to the present application and optionally an organic ligand compound selected from C4-C20 organic carboxylic acids,

the dispersion medium is selected from the group consisting of organic solvents, petroleum fractions or combinations thereof, the organic solvent is selected from the group consisting of aliphatic hydrocarbons, aromatic hydrocarbons, alicyclic hydrocarbons, halogenated hydrocarbons, alcohol solvents, ether solvents, ester solvents, ketone solvents or combinations thereof, the petroleum cut is selected from distillate oils with a distillation range of 150-524° C. or residual oil components with a boiling point >524° C.,

the activator is selected from the group consisting of elemental sulphur, sulphur-containing compounds, or combinations thereof.

In yet another aspect, there is provided the use of the unsupported catalyst composition of the present application in the hydro-upgrading of heavy oils.

In yet another aspect, the present application provides a process for the hydro-upgrading of heavy oils, comprising the step of subjecting a heavy oil feedstock to a hydro-upgrading reaction under heating conditions in the presence of hydrogen and the unsupported catalyst composition according to the present application, which is optionally presulphurized.

The unsupported hydrogenation catalyst and composition thereof according to the present application have higher oil phase dispersity and hydrogenation activity when used in hydrogenation reaction of hydrocarbons. The method for preparing the catalyst according to the present application has the advantages of small number of raw materials, simple preparation process, low material and energy consumption, and green and efficient preparation procedure.

In addition, the unsupported catalyst composition according to the present application also has oil solubility and high dispersibility, and further comprises a complex activator. When used in the hydrogenation and hydrocracking processes of heavy oils and residual oils, the composition can generate a nano-sized hydrogenation activity center in situ, significantly inhibit condensation reaction, promote asphaltene upgrading and improve system stability, and has the advantages of low coking rate, high residual oil conversion rate and asphaltene upgrading rate, and is beneficial to improving system stability and long-period stable operation of the equipment.

Other characteristics and advantages of the present application will be described in detail in the detailed description hereinbelow.

### BRIEF DESCRIPTION OF THE DRAWINGS

The drawings, forming a part of the present description, are provided to help the understanding of the present application, and should not be considered to be limiting. The present application can be interpreted with reference to the drawings in combination with the detailed description hereinbelow. In the drawings:

FIG. 1 shows the spectrum of a positive ion ESI high resolution mass spectrometry test of the catalyst obtained in Example 3;

FIG. 2 shows an IR spectrum of the catalyst obtained in Example 1;

FIG. 3 shows an IR spectrum of the catalyst obtained in Example 2;

FIG. 4 shows the IR spectra of the catalysts obtained in Examples 3 to 5; and

FIG. 5 is an image showing the results of dissolution of the catalyst obtained in Example 1 in diesel fuel;

FIG. 6 is an image showing the results of dissolution of the catalyst obtained in Example 3 in toluene.

### DETAILED DESCRIPTION OF THE INVENTION

The present application will be further described hereinafter in detail with reference to the drawings and specific embodiments thereof. It should be noted that the specific embodiments of the present application are provided for illustration purpose only, and are not intended to be limiting in any manner.

Any specific numerical value, including the endpoints of a numerical range, described in the context of the present application is not restricted to the exact value thereof, but should be interpreted to further encompass all values close to said exact value, for example all values within ±5% of said exact value. Moreover, regarding any numerical range described herein, arbitrary combinations can be made between the endpoints of the range, between each endpoint and any specific value within the range, or between any two specific values within the range, to provide one or more new numerical range(s), where said new numerical range(s) should also be deemed to have been specifically described in the present application.

Unless otherwise stated, the terms used herein have the same meaning as commonly understood by those skilled in the art; and if the terms are defined herein and their definitions are different from the ordinary understanding in the art, the definition provided herein shall prevail.

In the context of the present application, in addition to those matters explicitly stated, any matter or matters not mentioned are considered to be the same as those known in the art without any change. Moreover, any of the embodiments described herein can be freely combined with another one or more embodiments described herein, and the technical solutions or ideas thus obtained are considered as part of the original disclosure or original description of the present application, and should not be considered to be a new matter that has not been disclosed or anticipated herein, unless it is clear to the person skilled in the art that such a combination is obviously unreasonable.

All of the patent and non-patent documents cited herein, including but not limited to textbooks and journal articles, are hereby incorporated by reference in their entirety.

As described above, in the first aspect, the present application provides an unsupported hydrogenation catalyst, composed of a complex formed by bonding a metal central atom or central ion with an organic ligand through a coordination bond, wherein the metal is selected from the group consisting of Group VB metals, Group VIB metals, Group VIII metals, Group IB metals or combinations thereof that have a hydrogenation activity, the organic ligand comprises a hydrocarbyl moiety and a coordinating group consisted of carbon atom and oxygen atom, and forms a coordination bond with the metal central atom or central ion through an oxygen atom, wherein the catalyst shows an infrared spectrum with characteristic peaks at positions of 700-1000 cm⁻¹, 1350-1450 cm⁻¹ and 1500-1610 cm⁻¹.

According to the present application, the unsupported hydrogenation catalyst consists only of the complex and does not comprise any solid carrier component. However, if desired, the unsupported hydrogenation catalyst of the present application can also be present and used in the form of a composition with a liquid component capable of dispersing the catalyst, such as organic solvents and organic ligand compounds.

According to the present application, the Group VB metal, Group VIB metal, Group VIII metal and Group IB metal having a hydrogenation activity in the complex of the present application may be in the form of a central atom or a central ion, depending on the metal used.

In a preferred embodiment, the coordinating group may be —C(═O)—O group.

In a preferred embodiment, the catalyst has a schematic composition represented by formula (I):

MOₐ[R(COO)ₓ]_{b} (I),

wherein M represents the metal, R(COO)ₓ represents the organic ligand, R represents the hydrocarbyl moiety of the organic ligand, COO represents the coordinating group of the organic ligand, x represents the number of coordinating groups in the organic ligand, a represents the molar ratio of oxygen atom linked to the metal M via a non-coordination bond to the metal M, and b represents the molar ratio of the organic ligand to the metal M, wherein:

R is a C3-C19 hydrocarbyl group, preferably selected from the group consisting of C5-C11 normal alkyl, C5-C11 isomeric alkyl, C5-C12 alkyl with cycloalkyl moiety, C6-C12 aryl, or combinations thereof;

x represents the number of coordinating groups in the organic ligand, and is 1, 2 or 3, preferably 1 or 2;

a is a positive number from 0 to 3, preferably from 1 to 3; and

b is a positive number from 1 to 6, preferably from 2 to 5.

According to the present application, the unsupported hydrogenation catalyst may be a mixture of a plurality of different complexes, and the molar ratios a and b of the oxygen atom and the organic ligand to the metal M in the catalyst composition are calculated values based on analysis of the metal content and elemental composition, etc., and thus may be non-integers. When M is one type of metal, the molar ratios a and b are molar ratios relative to the metal; and when M is a combination of two or more metals, the molar ratios a and b are molar ratios relative to the total amount of all metals. In addition, the metal M (e.g. MoNi, MoCoV, etc.) shown in the catalyst composition only indicates which metals are present and does not indicate the molar ratio between the metals.

In some further preferred embodiments, at least a portion of the complex in the catalyst has a structure represented by formula (I-1):

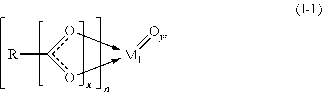

wherein M₁ represents a metal, and is one selected from the group consisting of Group VB metals, Group VIB metals, Group VIII metals and Group IB metals that have a hydrogenation activity;

→ represents a coordination bond;

R represents a C3-C19 hydrocarbyl group, and is preferably selected from the group consisting of C5-C11 normal alkyl, C5-C11 isomeric alkyl, C5-C12 alkyl with cycloalkyl moiety and C6-C12 aryl;

x represents the number of coordinating groups in the organic ligand, and is 1, 2 or 3, preferably 1 or 2;

n represents a coordination number, and is a positive number from 1 to 6, preferably from 2 to 5; and

y represents the number of oxygen atom linked to the metal M₁ via a non-coordination bond, and is a positive number from 0 to 3, preferably from 1 to 3.

In a further preferred embodiment, in the IR spectrum of the catalyst, the distance between a characteristic peak at the position of 1350-1450 cm⁻¹ and a characteristic peak at the position of 1500-1610 cm⁻¹ is less than 145 cm⁻¹. In some further preferred embodiments, at least a portion of the complex in the catalyst has a structure represented by formula (I-2):

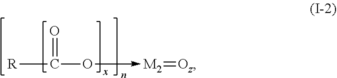

wherein M₂ represents a metal, and is at least two selected from the group consisting of Group VB metals, Group VIB metals, Group VIII metals, and Group IB metals that have a hydrogenation activity;

→ represents a coordination bond;

R represents a C3-C19 hydrocarbyl group, and is preferably selected from the group consisting of C5-C11 normal alkyl, C5-C11 isomeric alkyl, C5-C12 alkyl with cycloalkyl moiety and C6-C12 aryl;

x represents the number of coordinating groups in the organic ligand, and is 1 or 2, preferably 1;

n represents a coordination number, and is a positive number from 1 to 6, preferably from 2 to 5;

z represents the number of oxygen atom linked to the metal M₂ via a non-coordination bond, and is a positive number from 0 to 3, preferably from 1 to 3.

According to the present application, oxygen atom linked to the metal M₂ via a non-coordination bond in formula (I-2) includes both the oxygen atom linked to only one metal atom/ion in the complex molecule (e.g., the oxygen atom that forms an M═O bond) and the oxygen atom linked between two metal atoms/ions in the complex molecule (e.g., the oxygen atom that forms an M—O—M′ bond).

In a further preferred embodiment, in the IR spectrum of the catalyst, the distance between a characteristic peak at the position of 1350-1450 cm⁻¹ and a characteristic peak at the position of 1500-1610 cm⁻¹ is more than 145 cm⁻¹.

In a preferred embodiment, the Group VB metals, Group VIB metals, Group VIII metals and Group IB metals having a hydrogenation activity are selected from the group consisting of V, Cr, Mo, W, Fe, Co, Ru, Ni, Cu and Pd, more preferably selected from the group consisting of Mo, Ni, W, Fe, V and Co.

In the present application, the term “C3-C19 hydrocarbyl” refers to a hydrocarbyl group having 3 to 19 carbon atoms, which may be saturated or unsaturated, and may be normal hydrocarbyl, isomeric hydrocarbyl, or hydrocarbyl with cycloalkyl moiety, including, but not limited to, C3-C19 normal alkyl, C3-C19 isomeric alkyl, C5-C19 alkyl with cycloalkyl moiety, and C6-C19 aryl.

In the present application, the term “C3-C19 normal alkyl” refers to a normal alkyl group having 3 to 19 carbon atoms, preferably 5 to 11 carbon atoms, such as n-pentyl, n-hexyl, n-heptyl, n-octyl, n-nonyl, n-decyl, n-undecyl.

In the present application, the term “C3-C19 isomeric alkyl” refers to a isomeric alkyl group having 3 to 19 carbon atoms, preferably 5 to 11 carbon atoms, such as isopentyl, isohexyl, isoheptyl, isooctyl, isononyl, isodecyl, isoundecyl.

In the present application, the term “C5-C19 alkyl with cycloalkyl moiety” refers to a saturated hydrocarbyl group having 5 to 19 carbon atoms, preferably 5 to 12 carbon atoms, that comprises a saturated carbon ring, such as cyclopentyl, cyclohexyl, methylcyclohexyl, decahydronaphthyl, methyldecahydronaphthyl, ethyldecahydronaphthyl, and the like.

In the present application, the term “C6-C19 aryl” refers to a hydrocarbyl group having 6 to 19 carbon atoms that comprises an aromatic ring, such as phenyl, naphthyl, anthracyl, p-tolyl, benzyl, methylnaphthyl, methylanthracenyl, and the like, preferably an aryl group of 6 to 12 carbon atoms.

According to the present application, the C3-C19 hydrocarbyl, C3-C19 normal alkyl, C3-C19 isomeric alkyl, C5-C19 alkyl with cycloalkyl moiety, and C6-C19 aryl groups may be optionally substituted, e.g., may be unsubstituted, or may be substituted with one or more groups selected from halo, nitro, sulfonic acid group, and the like.

In a preferred embodiment, the organic ligand in the complex is derived from a C4-C20 organic carboxylic acid, preferably from one or more selected from the group consisting of C4-C20 normal or isomeric alkyl carboxylic acids, C6-C20 naphthenic carboxylic acids comprising a saturated carbon ring and C7-C20 aromatic carboxylic acids comprising an aromatic ring, more preferably from one or more selected from the group consisting of C6-C12 normal or isomeric alkyl carboxylic acids, C6-C13 naphthenic carboxylic acids comprising a saturated carbon ring and C7-C13 aromatic carboxylic acids comprising an aromatic ring, further preferably from one or more selected from the group consisting of succinic acid, hexanoic acid, adipic acid, heptanoic acid, octanoic acid, nonanoic acid, ethylhexanoic acid, oleic acid, petroleum acid, salicylic acid, benzoic acid and phenylacetic acid.

In a preferred embodiment, the unsupported hydrogenation catalyst has a metal content of from 5% to 35%, preferably from 8% to 30%, more preferably from 10% to 25%, particularly preferably from 10% to 20%, calculated based on metal and relative to the weight of the catalyst.

In a second aspect, the present application provides a method for preparing an unsupported hydrogenation catalyst, comprising the steps of:
1) mixing a metal source or a dispersion thereof with an organic ligand compound;
2) reacting the mixture obtained in step 1) for 1-8 h at 100-350° C.; and
3) collecting the resulting liquid product,

wherein the metal source is selected from the group consisting of elemental metal, metal oxide, metal hydroxide, metallic oxyacid, metal inorganic salt or combinations thereof, and the metal in the metal source is selected from the group consisting of Group VB metals, Group VIB metals, Group VIII metals, Group IB metals, and combinations thereof that have a hydrogenation activity;

the organic ligand compound is selected from the group consisting of C4-C20 organic carboxylic acids or anhydrides thereof; and

the molar ratio of the organic ligand compound to the metal in the metal source is 1-10:1.

In a preferred embodiment, the mixture obtained in step 1) is consisted of the metal source and the organic ligand compound; or is consisted of the metal source, a dispersion medium for dispersing the metal source, and the organic ligand compound.

In some particular embodiments, the method comprises the steps of:
i) dispersing a metal source into a dispersion medium to obtain a metal source dispersion;
ii) adding an organic ligand compound into the resulting metal source dispersion, heating to 100-350° C., and reacting for 1-8 h; and
iii) cooling after the completion of the reaction, and collecting the resulting liquid product.

In some other particular embodiments, the method comprises the steps of:
i) dispersing a metal source directly into an organic ligand compound;
ii) heating to 100-350° C. for reaction for 1-8 h; and
iii) cooling after the completion of the reaction, and collecting the resulting liquid product.

In a preferred embodiment, the metal source is selected from the group consisting of metal oxides, metal hydroxides, metal chlorides, metal sulphides, metal sulfates, metal nitrates, metal carbonates, metallic oxyacids, salts of metallic oxyacids, or combinations thereof, for example selected from the group consisting of oxides, hydroxides, chlorides, sulphides, sulfates, nitrates and carbonates of V, Mo, W, Fe, Co, Ni, Cu and Zn, molybdic acid, tungstic acid, various forms of ammonium molybdate, ammonium tungstate, or combinations thereof.

In a preferred embodiment, the organic ligand compound is selected from C4-C20 organic carboxylic acids, preferably selected from the group consisting of C4-C20 normal or isomeric alkyl carboxylic acids, C6-C20 naphthenic carboxylic acids comprising a saturated carbon ring, C7-C20 aromatic carboxylic acids comprising an aromatic ring, or combinations thereof.

According to the present application, the term “C4-C20 normal alkyl carboxylic acid” refers to a carboxylic acid having 4 to 20 carbon atoms obtained by linking one or more carboxyl groups to a normal alkane, such as butyric acid, succinic acid, valeric acid, hexanoic acid, adipic acid, heptanoic acid, octanoic acid, nonanoic acid, ethylhexanoic acid, tridecanoic acid, oleic acid, and the like.

According to the present application, the term “C4-C20 isomeric alkyl carboxylic acid” refers to a carboxylic acid having 4 to 20 carbon atoms obtained by linking one or more carboxyl groups to a isomeric alkane, such as isobutyric acid, isovaleric acid, isohexanoic acid, ethylhexanoic acid.

According to the present application, the term “C6-C20 naphthenic carboxylic acid comprising a saturated carbon ring” refers to a carboxylic acid having 6 to 20 carbon atoms obtained by linking one or more carboxyl groups to an alkane compound comprising a saturated carbon ring, such as cyclohexanecarboxylic acid, cyclohexanedicarboxylic acid, decahydro-naphthoic acid, decahydro-naphthoic dicarboxylic acid.

According to the present application, the term “C7-C20 aromatic carboxylic acid comprising an aromatic ring” refers to a carboxylic acid having 7 to 20 carbon atoms obtained by linking one or more carboxyl groups to an aromatic hydrocarbon, i.e., a hydrocarbon compound comprising an aromatic ring, such as benzoic acid, phenylacetic acid, phthalic acid, phenylpropionic acid.

In a further preferred embodiment, the organic ligand compound is selected from the group consisting of C6-C12 normal or isomeric alkyl carboxylic acids, C6-C13 naphthenic carboxylic acids comprising a saturated carbon ring, C7-C13 aromatic carboxylic acids comprising an aromatic ring or combinations thereof, even more preferably selected from the group consisting of succinic acid, hexanoic acid, adipic acid, heptanoic acid, octanoic acid, nonanoic acid, ethylhexanoic acid, oleic acid, petroleum acid, salicylic acid, benzoic acid, phenylacetic acid or combinations thereof.

In a preferred embodiment, the dispersion medium in the metal source dispersion may be an inorganic dispersion medium or an organic dispersion medium, and the inorganic dispersion medium may be selected from the group consisting of water, carbonic acid, hydrochloric acid, sulfuric acid, or phosphoric acid; the organic dispersion medium may be selected from the group consisting of aliphatic hydrocarbons, aromatic hydrocarbons, alicyclic hydrocarbons, halogenated hydrocarbons, alcohol solvents, ether solvents, ester solvents, ketone solvents, or combinations thereof, and more preferably selected from the group consisting of ethanol, toluene, xylene, petroleum ether, gasoline, diesel oil, or combinations thereof.

In a further preferred embodiment, the weight ratio of the dispersion medium to the metal source in the dispersion is 1-25:1, more preferably 2-8:1.

In a preferred embodiment, in step 2), the reaction temperature is 160-260° C. and the reaction time is 2-5 h.

According to the present application, the pressure and reaction atmosphere used in step 2) are not particularly limited, for example, the reaction pressure may be atmospheric pressure, and the reaction atmosphere may be air, nitrogen or an inert atmosphere.

In a third aspect, the present application provides a hydrogenation catalyst composition, comprising the unsupported hydrogenation catalyst according to the present application and at least one organic ligand compound and/or at least one organic solvent, wherein the organic ligand compound is selected from C4-C20 organic carboxylic acids.

In a preferred embodiment, the organic ligand compound is selected from the group consisting of C4-C20 normal or isomeric alkyl carboxylic acids, C6-C20 naphthenic carboxylic acids comprising a saturated carbon ring, C7-C20 aromatic carboxylic acids comprising an aromatic ring or combinations thereof, preferably selected from the group consisting of C6-C12 normal or isomeric alkyl carboxylic acids, C6-C13 naphthenic carboxylic acids comprising a saturated carbon ring, C7-C13 aromatic carboxylic acids comprising an aromatic ring or combinations thereof, more preferably selected from the group consisting of succinic acid, hexanoic acid, adipic acid, heptanoic acid, octanoic acid, nonanoic acid, ethylhexanoic acid, oleic acid, petroleum acid, salicylic acid, benzoic acid, phenylacetic acid or combinations thereof.

The organic solvent is not particularly limited in the present application as long as it can disperse or be miscible with the unsupported hydrogenation catalyst, and may be, for example, aliphatic hydrocarbons, aromatic hydrocarbons, alicyclic hydrocarbons, halogenated hydrocarbons, alcohol solvents, ether solvents, ester solvents, ketone solvents or combinations thereof, preferably selected from the group consisting of toluene, gasoline, ethanol, diesel oil, or combinations thereof.

In a preferred embodiment, the unsupported hydrogenation catalyst is present in an amount of from 50% to 95%, preferably from 80% to 95%; and the total amount of the organic ligand compound and the organic solvent is from 5% to 50%, preferably from 5% to 20%, based on the weight of the composition.

In a preferred embodiment, the composition comprises at least one organic ligand compound, and the composition shows an infrared spectrum with characteristic peaks at positions of 700-1000 cm⁻¹, 1350-1450 cm⁻¹, 1500-1610 cm⁻¹ and 1700-1750 cm⁻¹, wherein the characteristic peaks at positions of 700-1000 cm⁻¹, 1350-1450 cm⁻¹ and 1500-1610 cm⁻¹ are characteristic peaks of the metal-organic complex, and the characteristic peak at the position of 1700-1750 cm⁻¹ is a characteristic peak of the organic ligand compound.

In a further preferred embodiment, the hydrogenation catalyst composition is consisted of the unsupported hydrogenation catalyst and at least one organic ligand compound. In this case, the composition of the hydrogenation catalyst composition may also be expressed schematically by using the formula (I), MOₐ[R(COO)ₓ]_{b}, wherein M, a, R and x are as defined above, and b represents the molar ratio of the total amount of organic ligand and organic ligand compound to the metal M.

In some particular embodiments, the hydrogenation catalyst composition may further comprise other components for improving oil solubility, storage stability, and oxidation resistance, for example, organic materials having a capability of reducibility and stability such as formic acid, oxalic acid, formaldehyde, ethylenediamine, oleylamine, and the like, and said other components may be present in an amount of 0% to 80%, preferably 0% to 50%, by weight of the composition.

In a fourth aspect, there is provided the use of the unsupported hydrogenation catalyst or hydrogenation catalyst composition according to the present application in the hydrogenation of hydrocarbons.

In a fifth aspect, there is provided a process for hydroprocessing a hydrocarbonaceous feedstock, comprising the step of contacting the hydrocarbonaceous feedstock with the unsupported hydrogenation catalyst or hydrogenation catalyst composition according to the present application for a hydrogenation reaction.

According to the present application, the hydrocarbonaceous feedstock can be various unsaturated hydrocarbon compounds Is such as benzene, alkylbenzenes, naphthalenes, alkylnaphthalenes, anthracenes, alkyl anthracenes, and the like; or mixtures of unsaturated hydrocarbon compounds, such as crude oil, gasoline, diesel oil, vacuum gas oil, residual oil, and the like.

In a preferred embodiment, the conditions for the hydrogenation reaction include a reaction temperature of 380-430° C., an initial hydrogen pressure of 5-20 MPa, a fresh feed liquid hourly space velocity of 0.05-1.0 h⁻¹, and a catalyst concentration (calculated based on metal) of 50-10000 μg/g relative to the total feed.

In a sixth aspect, the present application provides an unsupported catalyst composition comprising, by weight, from 10% to 45% of a hydrogenation catalyst component, from 45% to 80% of a dispersion medium and from 1.0% to 10% of an activator, wherein the hydrogenation catalyst component is consisted of the unsupported hydrogenation catalyst of the present application and optionally an organic ligand compound, wherein the organic ligand compound is selected from C4-C20 organic carboxylic acids.

According to the present application, the C4-C20 organic carboxylic acids that may be comprised in the hydrogenation catalyst component as the organic ligand compound may be those specifically described in the first aspect of the present application, of which the detailed description will be omitted herein.

According to the present application, the dispersion medium suitable for use in the unsupported catalyst composition can be any liquid material capable of enhancing the dissolution, dispersion of the metal-organic complex in the unsupported hydrogenation catalyst, including but not limited to organic solvents and petroleum fractions capable of dispersing the catalyst or being miscible with the catalyst. The organic solvent may be selected from the group consisting of aliphatic hydrocarbons, aromatic hydrocarbons, alicyclic hydrocarbons, halogenated hydrocarbons, alcohol solvents, ether solvents, ester solvents, ketone solvents, or combinations thereof, preferably selected from the group consisting of aliphatic hydrocarbons, aromatic hydrocarbons, alicyclic hydrocarbons, or combinations thereof, such as n-octane, cyclohexane, toluene, and decahydronaphthalene, and more preferably an aromatic hydrocarbon solvent. The petroleum fraction may be selected from distillate oils having a distillation range of 150-524° C. or residual oil components having a boiling point >524° C., such as solvent gasoline, AGO fractions, LCO, oil slurry, furfural extract, atmospheric residuum, and vacuum residuum, preferably an aromatic-rich petroleum fration.

According to the present application, the activator suitable for the unsupported catalyst composition is a material capable of activating the M-O bond in the metal-organic complex of the unsupported hydrogenation catalyst to form a M-S bond in the hydrogenation active phase, and can be, for example, elemental sulfur, sulfur-containing compounds, mixtures of sulfur-containing compounds, or combinations thereof, preferably selected from the group consisting of thiols, thioethers, carbon disulphide, sulfur, thiopheneic compounds, or combinations thereof.

In a preferred embodiment, the hydrogenation catalyst component is present in an amount of from 10% to 45%, preferably from 10% to 30%, the dispersion medium is present in an amount of from 45% to 80%, preferably from 60% to 80%, and the activator is present in an amount of from 1.0% to 10%, preferably from 3.0% to 10.0%, based on the weight of the unsupported catalyst composition.

In a seventh aspect, there is provided the use of the unsupported catalyst composition of the present application in the hydro-upgrading of heavy oils.

In an eighth aspect, the present application provides a process for the hydro-upgrading of heavy oils, comprising the step of subjecting a heavy oil feedstock to a hydro-upgrading reaction under heating conditions in the presence of hydrogen and the unsupported hydrogenation catalyst composition of the present application, which is optionally presulphurized.

In a preferred embodiment, the conditions of the hydro-upgrading include: an amount of the unsupported catalyst composition of 50-10000 μg/g, preferably 50-3000 μg/g, calculated based on metal and relative to the weight of the heavy oil feedstock; an initial hydrogen pressure of 5-20 MPa, preferably 5-15 MPa; a reaction temperature of 360-480° C., preferably 390-450° C.; a liquid hourly space velocity of 0.05-2.0 h⁻¹, preferably 0.05-1.0 h⁻¹; and a hydrogen-to-oil volume ratio of 300-2000, preferably 500-1500.

### EXAMPLES

The present application will be further illustrated with reference to the following examples, but the present application is not limited thereto.

In the following examples, unless otherwise specified, reagents and raw materials used are commercially available products, which are chemically pure.

In the following examples, the metal content of the resulting product was measured by Inductively Coupled Plasma Optical Emission Spectrometry (ICP-OES) using a SPECTRO ARCOS SOP plasma emission spectrometer under the conditions that an optical chamber was sealed and filled with argon gas, and the measurement was performed under vertical observation at a wavelength of 130-770 nm.

In the following examples, the elemental composition of the resulting product was determined as follows: the content of element C, H was determined using Cara Erba EA1110 elemental analyzer, Italy, according to the SH0656 method; the content of element S was measured according to the energy dispersion X fluorescence spectrometry GB 17040 method; and the content of element O was measured by O-content method.

In the following examples, the spectrum of the positive ion ESI high resolution mass spectrometry test of the resulting products were measured by a fourier transform ion cyclotron resonance mass spectrometer (FT-ICR MS) of Bruker under the following conditions: magnetic field strength 15 T, electrospray ionization source, negative ion mode.

In the following examples, the IR spectra of the resulting products were measured using a NICOLET IS50 spectrometer of Thermo Fisher with a scanning wavelength of 400-4000 cm⁻¹ and 16 times of scanning. The ZnSe crystal and the HgCdTe infrared detector were used together to measure the Attenuated Total Reflectance (ATR) of the sample, with a resolution of 4 cm⁻¹.

### Examples 1 to 7

The compounds were weighed in the amounts shown in Table 1, placed in a three-necked flask, and reacted under the conditions shown in Table 1. After completion of the reaction, the metal compounds in the flasks of Examples 1 to 5 were completely dissolved, while unreacted metal compounds were remained in the flasks of Examples 6 to 7. The liquid reaction products in the flasks obtained in Examples 1 to 5 were poured out to obtain a target product; the products of Examples 6 to 7 were filtered to remove unreacted metal compounds, and liquid products were obtained as a target product. The metal contents of the resulting products were measured using inductively coupled plasma optical emission spectrometry (ICP-OES), and the elemental composition of the resulting products was measured according to a corresponding method, and the composition of the products was determined according to the measured results of the metal content and elemental composition. The raw materials and reaction conditions used and the test results of Examples 1 to 7 are shown in Table 1.

TABLE 1
Raw materials, reaction conditions and results of the examples

| Examples | Example 1* | Example 2* | Example 3* | Example 4* | Example 5* | Example 6* | Example 7** |
|---|---|---|---|---|---|---|---|
| Raw materials | Ammonium | Ferric nitrate | Ammonium | Ammonium | Ammonium molybdate | Ammonium | Ammonium |
| and amounts | molybdate | (40.4g) | molybdate | molybdate | (8.83 g) | molybdate | molybdate |
| thereof | (17.66 g) | | (8.87 g) | (8.83 g) | | (17.66 g) | (8.87 g) |
| | Ethyl | Formic acid | Nickel nitrate | Cobalt nitrate | Ammonium metavanadate | Ethyl | Nickel nitrate |
| | hexanoic acid | (9.2 g) | (14.5 g) | (14.6 g) | (5.85 g) | hexanoic acid | (14.5 g) |
| | (43.2 g) | | | | | (30.5 g) | |
| | — | Ethyl | Formic acid | Acetic acid (12 | Ferric nitrate (10.1 g) | — | Formic acid |
| | | hexanoic acid | (27.6 g) | g) | | | (27.6 g) |
| | | (43.2 g) | | | | | |
| | — | — | Ethyl | Ethyl hexanoic | Oxalic acid (34.02 g) | — | Ethyl |
| | | | hexanoic acid | acid (43.2 g) | | | hexanoic acid |
| | | | (43.2 g) | | | | (14.5 g) |
| | — | — | — | — | Petroleum acid (37.8 g) | — | — |
| Reaction | 210 | 210 | 200 | 240 | 190 | 210 | 200 |
| temperature/° | | | | | | | |
| C. | | | | | | | |
| Reaction | Air | Air | Air | Air | Air | Air | Air |
| atmosphere | | | | | | | |
| Reaction | Atmospheric | Atmospheric | Atmospheric | Atmospheric | Atmospheric pressure | Atmospheric | Atmospheric |
| pressure | pressure | pressure | pressure | pressure | | pressure | pressure |
| Reaction | 5 | 3 | 4 | 3 | 6 | 5 | 4 |
| time/h | | | | | | | |
| Metal content | 18.8 | 13.27 | 15.74 | 16.71 | 12.75 | 20.4 | 33.3 |
| of the | | | | | | | |
| product/% | | | | | | | |
| Composition of the product*** | MoO(i- C₇H₁₆COO)_{2.88} | FeO_{1.7}(i- C₇H₁₆COO)_{2.56} | (MoNi)O1.0(i- C₇H₁₆COO)_{2.67} | (MoCo)O_{1.2}(i- C₇H₁₆COO)_{2.80} | 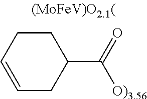 | MOO_{1.6}(i- C₇H₁₆COO)_{2.0} | (MoNi)O_{1.2}(i- C₇HCOO)_{2.0} |

Note:
*The metal compounds were completely dissolved in Examples 1 to 5, indicating that the organic ligand compounds might be used in an excessive amount, and thus the resulting products are unsupported hydrogenation catalysts or hydrogenation catalyst compositions of the present application;
**In Examples 6 to 7, unreacted metal compounds were remained, indicating that the metal compounds were used in an excessive amount, and thus the resulting products are unsupported hydrogenation catalysts of the present application;
***The metal (e.g. MoNi, MoCo, MoFeV) shown in the composition of the resulting products only shows which metals are present while does not indicate the molar ratio between the metals.

As shown in Table 1, the metal content of the unsupported hydrogenation catalyst or hydrogenation catalyst composition of the present application can be 12.75% to 33.3%, and the molar ratio of the organic ligand to the metal in the catalyst or the molar ratio of the total of organic ligand and organic ligand compound to the metal in the catalyst composition is 2.0 to 3.56.

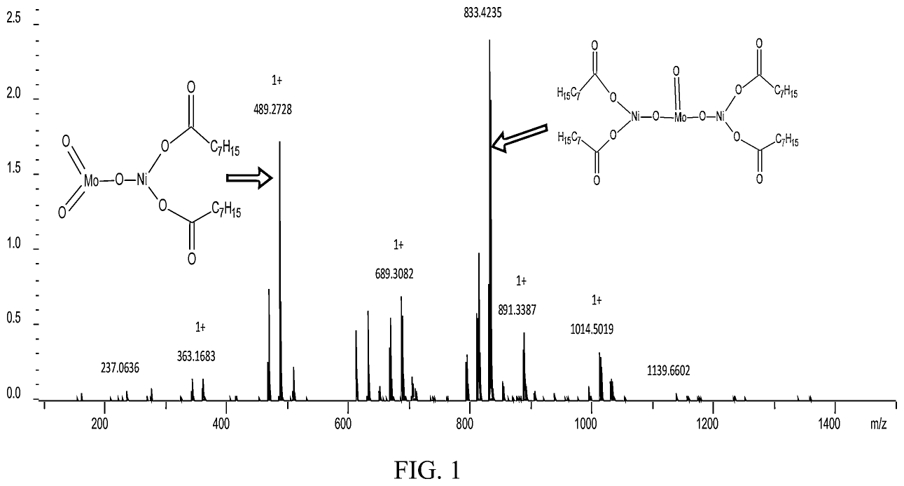

The spectrum of the positive ion ESI high resolution mass spectrometry test of the product obtained in Example 3 was measured by FT ICR MS analysis to determine the molecular weight distribution of molybdenum-nickel-ethyl hexanoic acid complexes in the resulting product, of which the results are shown in FIG. 1. As can be seen from the data fitting of the molecular weight, the product obtained in Example 3 comprises both the molybdenum-nickel-ethyl hexanoic acid complex (MoNi)O₃(i-C₇H₁₆COO)₂ with a coordination number of 2 and the molybdenum-nickel-ethyl hexanoic acid complex (MoNi)O₃(i-C₇H₁₆COO)₄ with a coordination number of 4.

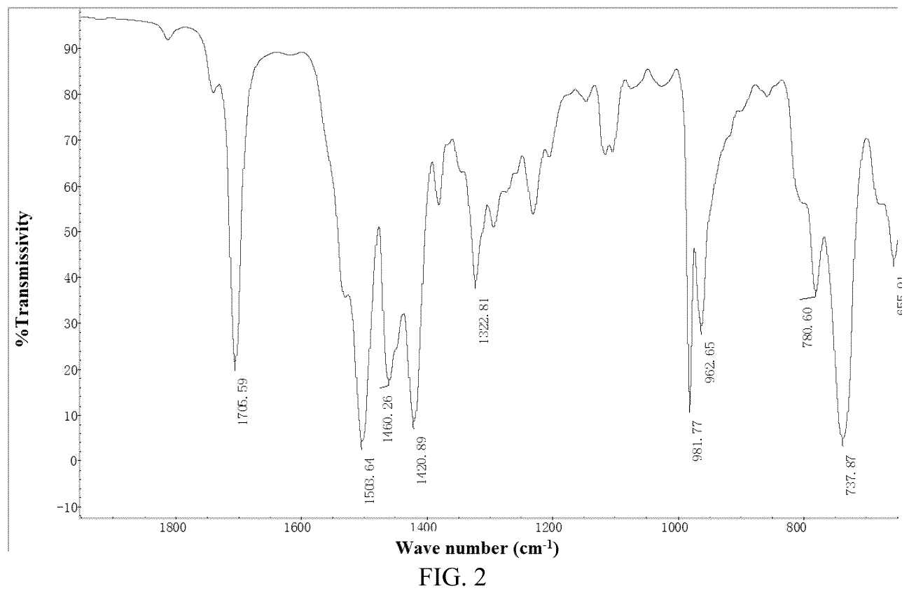

The IR spectra of the products obtained in Examples 1 to 5 are shown in FIGS. 2-4. As can be seen from FIGS. 2-4, the products of all those examples show characteristic peaks at positions of 700-1000 cm⁻¹, 1350-1450 cm⁻¹ and 1500-1610 cm⁻¹.

As shown in FIG. 2, the product of Example 1 shows M-O, M═O vibrational characteristic peaks at the position of 700-1000 cm⁻¹ and characteristic peaks of the coordination between the —C(═O)—O group and the metal at positions of 1350-1450 cm⁻¹ and 1500-1610 cm⁻¹, and the distance between a characteristic peak at the position of 1350-1450 cm⁻¹ and a characteristic peak at the position of 1500-1610 cm⁻¹ (i.e. the difference of the wave numbers corresponding to the positions of the crest of those peaks) is less than 145 cm⁻¹, which indicates that a complex with a monometallic bidentate coordination structure represented by the following formula is present in the product:

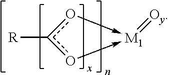

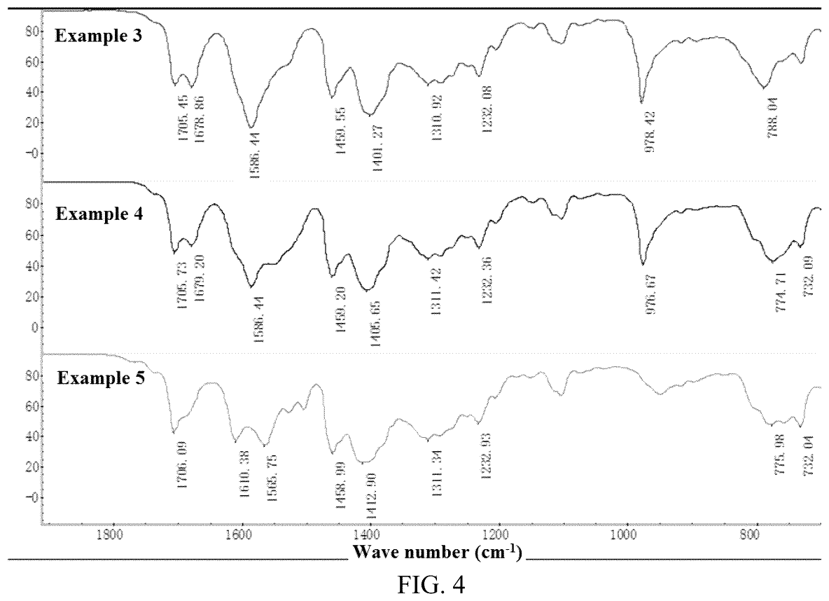

As shown in FIG. 4, the catalysts of Examples 3-5 show M-O, M═O vibrational characteristic peaks at the position of 700-1000 cm⁻¹, and characteristic peaks of the coordination between the —C(═O)—O group and the metal at positions of 1350-1450 cm⁻¹ and 1500-1610 cm⁻¹, and the distance between a characteristic peak at the position of 1350-1450 cm⁻¹ and a characteristic peak at the position of 1500-1610 cm⁻¹ is more than 145 cm⁻¹, indicating that a complex with a bimetallic or polymetallic monodentate coordination structure represented by the following formula is present in the product:

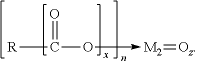

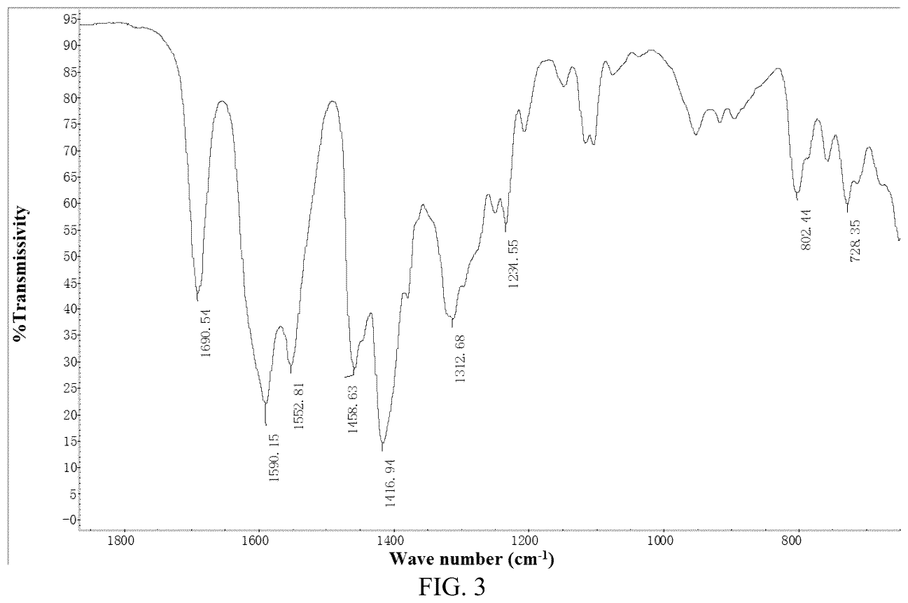

As shown in FIG. 3, the catalyst obtained in Example 2 shows characteristic peaks of coordination between the —C(═O)—O group and the metal at positions of 1350-1450 cm⁻¹ and 1500-1610 cm⁻¹, in which two characteristic peaks are present at the position of 1500-1610 cm⁻¹, the distance between one of the two characteristic peaks and the characteristic peak at the position of 1350-1450 cm⁻¹ is less than 145 cm⁻¹, and the distance between the other of the two characteristic peaks and the characteristic peak at the position of 1350-1450 cm⁻¹ is more than 145 cm⁻¹, indicating that both a complex with a bidentate coordination structure and a complex with a monodentate coordination structure are present in the catalyst, of which the structural formulas are respectively shown above.

### Examples 8 to 9

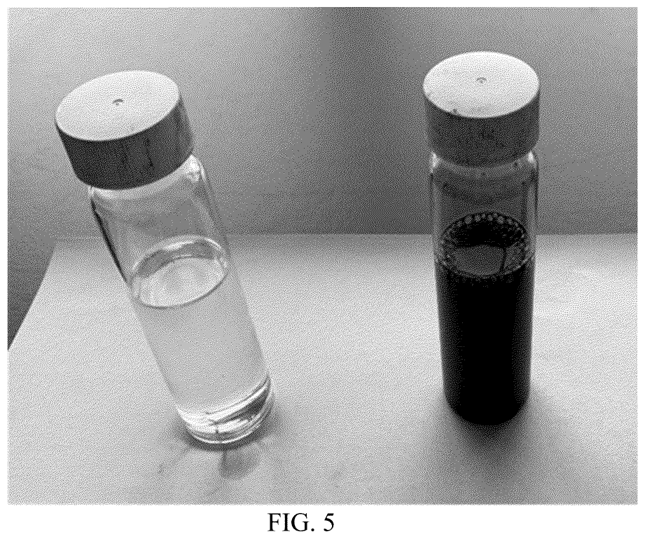

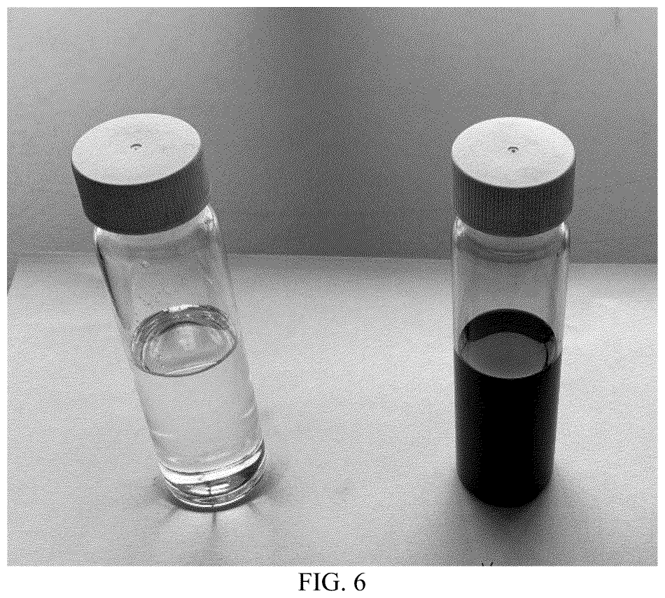

The catalyst products obtained in Example 1 and Example 3 were dispersed in diesel oil and toluene, respectively, to evaluate the oil solubility of the unsupported hydrogenation catalyst obtained by the method of the present application. The dissolution and dispersion results of the products of Example 1 and Example 3 in diesel oil and toluene are shown in FIGS. 5 and 6, respectively, wherein the diesel oil and toluene before dissolution are shown on the left side of the figure, and the diesel oil after dissolution of the product of Example 1 and the toluene after dissolution of the product of Example 3 are shown on the right side of the figure. As can be seen from FIGS. 5 and 6, the unsupported hydrogenation catalyst synthesized according to the present application is completely miscible with diesel oil and toluene, indicating that the unsupported hydrogenation catalyst has good oil solubility.

### Examples 10 to 11

The products obtained in Examples 1 and 5 were used as catalysts for the hydrogenation of an aromatic hydrocarbon, i.e. pyrene, wherein tetralin was used as a solvent, the mass fraction of pyrene in 10 g of total reactants (pyrene+solvent) was 10%, and the reaction was carried out in a 100 ml continuously stirred autoclave under test conditions including an initial hydrogen pressure of 9 MPa, a reaction temperature of 420° C., a reaction time of 180 min, and a catalyst concentration (calculated based on metal) of 2500 μg/g relative to the weight of total reactants. The test results are shown in Table 2.

### Comparative Example 1

The test was conducted as described in Example 10 except that the product obtained in Example 1 was replaced with a conventional supported catalyst (Ni—Mo supported catalyst for hydrogenation of residual oil, with a Mo content of 9.3% by mass, a Ni content of 2.52% by mass) in an equivalent substitution manner in terms of the amount of the metal. The test results are shown in Table 2.

TABLE 2
Results of Examples 10 to 11 and Comparative example 1

| | | | Comparative |
|---|---|---|---|
| Item | Example 10 | Example 11 | Example 1 |
| Catalyst | Product of | Product of | Ni—Mo supported |
| | Example 1 | Example 5 | catalyst |
| Test results | | | |
| Pyrene conversion/% | 71.24 | 78.32 | 70.87 |
| Product distribution/% | | | |
| Dihydropyrene | 0 | 5.31 | 23.11 |
| Tetrahydropyrene | 10.61 | 5.31 | 5.71 |
| Hexahydropyrene + | 60.63 | 52.11 | 42.05 |
| decahydropyrene | | | |
| Hydrogen | 11.62 | 10.63 | 8.87 |
| consumption/mmol | | | |

From the results of Table 2, it can be seen that the unsupported hydrogenation catalyst and hydrogenation catalyst composition of the present application show higher conversion rate of pyrene and yield of deep-hydrogenated product, and higher hydrogen consumption, compared to the supported catalyst, indicating that the catalyst of the present application has higher catalytic activity.

### Examples 12 to 14

The product obtained in Example 1 and an organic ligand compound (ethyl hexanoic acid) were formulated into a composition at a mass ratio of 95:5, the composition, the product obtained in Example 3 and the product obtained in Example 6 were respectively used as catalysts to catalyze the hydrocracking reaction of an alkyl substituted aromatic hydrocarbon, i.e. dodecyl pyrene, wherein decahydronaphthalene was used as a solvent, the mass fraction of dodecyl pyrene in 10 g of total reactants (dodecyl pyrene+solvent) was 10%, the reaction was carried out in a 100 ml autoclave under the test conditions including an initial hydrogen pressure of 9 Mpa, a reaction temperature of 420° C., a reaction time of 60 min, and a catalyst concentration (calculated based on metal) of 2500 μg/g, relative to the weight of the total reactants. The test results are shown in Table 3.

TABLE 3
Results of Examples 12 to 14

| Item | Example 12 | Example 13 | Example 14 |
|---|---|---|---|
| Catalyst | 95% of the product | Product of | Product of |
| | of Example 1 + 5% | Example 3 | Example 6 |
| | of organic ligand | | |
| | compound | | |
| Results of dodecyl | | | |
| pyrene test | | | |
| Conversion of | 100 | 100 | 97 |
| cracking/% | | | |
| Condensation rate/% | 0 | 0 | 0 |
| Hydrogen | 2.34 | 1.38 | 1.26 |
| consumption/mmol | | | |

From the results in Table 3, it can be seen that both the unsupported hydrogenation catalyst of the present application and its composition achieve a conversion of dodecyl pyrene cracking of >97% and a dodecyl pyrene condensation rate of zero; compared with the unsupported hydrogenation catalyst product of Example 6, the catalyst composition comprising or being further added with the organic ligand compound shows higher dodecyl pyrene conversion rate and hydrogen consumption, indicating that the catalyst composition of the present application has better coke inhibiting and cracking promoting activity compared with the catalyst itself.

### Examples 15 to 17 and Comparative Example 2

A residual oil catalytic hydro-thermal conversion test was carried out in a 2 L batch autoclave under the catalyst and reaction conditions shown in Table 4, using 200 g of vacuum residuum A having an asphaltene content of 14%, a Conradson carbon residue value of 26.4%, and a heavy metal (Ni+V) content of 210 μg/g as a feedstock. The test results are shown in Table 4. Table 4 Results of

### Examples 15 to 17 and Comparative Example 2

| | Example | Example | Example | Comparative |
|---|---|---|---|---|
| Item | 15 | 16 | 17 | Example 2 |
| Catalyst | 90% of the | Product of | Product of | Supported |
| | product of | Example 4 | Example 5 | catalyst of |
| | Example 3 + | | | Comparative |
| | 10% of organic | | | Example 1 |
| | ligand | | | |
| | compound | | | |
| Reaction | 430 | 430 | 425 | 430 |
| temperature/° C. | | | | |
| Reaction time/ | 300 | 300 | 130 | 300 |
| min | | | | |
| Initial hydrogen | 9 | 9 | 9 | 9 |
| pressure/MPa | | | | |
| The amount of | 3000 | 2000 | 2000 | 2000 |
| catalyst | | | | |
| (calculated | | | | |
| based on | | | | |
| metal)/(μg/g) | | | | |
| Cracking rate of | 91.40 | 90.04 | 65.91 | 84.72 |
| residual oil/% | | | | |
| Coke rate/% | 0.67 | 1.95 | 0.72 | 3.87 |
| Yield of | 86.82 | 84.27 | 55.83 | 78.20 |
| distillate oil/% | | | | |

As can be seen from the results of Table 4, the unsupported hydrogenation catalyst of the present application and its composition show higher cracking rate of residual oil, lower coke rate and higher yield of distillate oil under the same reaction conditions as compared to the conventional supported catalyst.

### Examples 18 to 20

Unsupported hydrogenation catalyst compositions were formed using the catalyst products of Examples 1 and 6, dispersion mediums and activators, and the content of each component in the unsupported hydrogenation catalyst compositions obtained are shown in Table 5.

TABLE 5
Composition of the catalyst compositions
obtained in Examples 18-20

| Item | Example 18 | Example 19 | Example 20 |
|---|---|---|---|
| Hydrogenation | Product of | Product of | Product of |
| catalyst | Example 1, 14% | Example 1, 37% | Example 6, 40% |
| component and | | | |
| content thereof | | | |
| (wt %) | | | |
| Dispersion | LCO, 81% | Furfural extract, | Furfural extract, |
| medium and | | 53% | 50% |
| content thereof | | | |
| (wt %) | | | |
| Activator and | DMDS, 5% | Sulfur powder, | Sulfur powder, |
| content thereof | | 10% | 10% |
| (wt %) | | | |

### Example 21

The catalyst composition obtained in Example 19 was subjected to a presulphurization treatment at a reaction temperature of 360° C., an initial hydrogen pressure of 5 MPa, and a reaction time of 30 min, and the reaction product was collected after the completion of the test.

### Examples 22 to 25

The catalyst composition products of Examples 19, 20 and 21 and the catalyst product of Example 1 were mixed with 200 g of a vacuum residuum B feedstock having an asphaltene content of 12.8%, a Conradson carbon residue value of 26.3% and a heavy metal (Ni+V) content of 220 μg/g, respectively, and subjected to a vacuum residuum hydro-thermal conversion test in a 2 L batch autoclave with stirring at a reaction temperature of 425° C., an initial hydrogen pressure of 9 MPa and a reaction time of 130 min. The test results are shown in Table 6.

TABLE 6
Reaction conditions and results of Examples 22-25

| Residual oil | | | | |
|---|---|---|---|---|
| hydro-thermal | Exam- | Exam- | Exam- | Exam- |
| conversion test | ple 22 | ple 23 | ple 24 | ple 25 |
| Catalyst | Product of | Product of | Product of | Product of |
| | Exam- | Exam- | Exam- | Exam- |
| | ple 19 | ple 1 | ple 20 | ple 21 |
| Amount of catalyst | 1500 | 1500 | 1500 | 1500 |
| added/(calculated | | | | |
| based on metal, | | | | |
| μg/g) | | | | |
| Reaction | 425 | 425 | 425 | 425 |
| temperature/° C. | | | | |
| Initial hydrogen | 9 | 9 | 9 | 9 |
| pressure/MPa | | | | |
| Reaction time/min | 130 | 130 | 130 | 130 |
| Reaction results | | | | |
| Conversion of | 67.78 | 64.10 | 65.84 | 68.42 |
| residual oil/% | | | | |
| Coke rate/% | 0.63 | 1.21 | 0.88 | 0.50 |
| Asphaltene | 82.68 | 61.66 | 68.72 | 84.23 |
| upgrading Rate/% | | | | |

From a comparison between the results of Example 23 and Example 22 shown in Table 6, it can be seen that the unsupported catalyst composition product of Example 19 comprising an activator shows a slightly higher residual oil conversion rate, a higher asphaltene upgrading rate and a lower coke rate, and the condensation rate is reduced by 48%, compared to the product of Example 1 free of the activator, indicating that it has a better performance for inhibiting asphaltene condensation reaction.

Meanwhile, as can be seen from a comparison between the results of Example 24 and Example 22 shown in Table 6, the unsupported catalyst composition product of Example 19, which comprises an organic ligand compound (i.e., ethylhexanoic acid), shows a higher residual oil conversion rate and asphaltene upgrading rate, and a lower coke rate, as compared to the unsupported catalyst composition product of Example 20, which does not comprise the organic ligand compound.

In addition, as can be seen from a comparison between the results of Example 25 and Example 22 shown in Table 6, where the unsupported catalyst composition product of Example 19 has been subjected to a presulphurization treatment, the residual oil conversion rate and asphaltene upgrading rate can be further improved, and the coke rate can be further reduced.

The present application is illustrated in detail herein above with reference to preferred embodiments, but is not intended to be limited to those embodiments. Various modifications may be made following the inventive concept of the present application, and these modifications shall be within the scope of the present application.

It should be noted that the various technical features described in the above embodiments may be combined in any suitable manner without contradiction, and in order to avoid unnecessary repetition, various possible combinations are not described in the present application, but such combinations shall also be within the scope of the present application.

In addition, the various embodiments of the present application can be arbitrarily combined as long as the combination does not depart from the spirit of the present application, and such combined embodiments should be considered as the disclosure of the present application.

1. An unsupported hydrogenation catalyst, composed of a complex formed by bonding a metal central atom or central ion with an organic ligand through a coordination bond, wherein the metal is selected from the group consisting of Group VB metals, Group VIB metals, Group VIII metals, Group IB metals or combinations thereof that have a hydrogenation activity, the organic ligand comprises a hydrocarbyl moiety and a coordinating group that is —C(═O)—O group, and forms a coordination bond with the metal central atom or central ion through an oxygen atom, and the catalyst shows an infrared spectrum with characteristic peaks at positions of 700-1000 cm⁻¹, 1350-1450 cm⁻¹ and 1500-1610 cm⁻¹.

2. The unsupported hydrogenation catalyst according to claim 1, wherein the catalyst has a schematic composition represented by formula (I):
MOₐ[R(COO)ₓ]_{b} (I),
wherein M represents the metal, R (COO) x represents the organic ligand, R represents the hydrocarbyl moiety of the organic ligand, COO represents the coordinating group of the organic ligand, x represents the number of coordinating groups in the organic ligand, a represents the molar ratio of oxygen atom linked to the metal M via a non-coordination bond to the metal M, and b represents the molar ratio of the organic ligand to the metal M, wherein:
R is a C3-C19 hydrocarbyl group, preferably selected from the group consisting of C5-C11 normal alkyl, C5-C11 isomeric alkyl, C5-C12 alkyl with cycloalkyl moiety, C6-C12 aryl, or combinations thereof;
x is 1, 2 or 3, preferably 1 or 2;
a is a positive number from 0 to 3, preferably from 1 to 3; and
b is a positive number from 1 to 6, preferably from 2 to 5.

3. The unsupported hydrogenation catalyst according to claim 1, wherein at least a portion of the complex in the catalyst has a structure represented by formula (I-1): 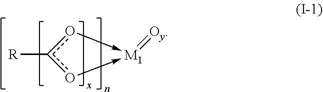
wherein M₁ represents a metal, and is one selected from the group consisting of Group VB metals, Group VIB metals, Group VIII metals and Group IB metals that have a hydrogenation activity;
→ represents a coordination bond;
R represents a C3-C19 hydrocarbyl group, and is preferably selected from the group consisting of C5-C11 normal alkyl, C5-C11 isomeric alkyl, C5-C12 alkyl with cycloalkyl moiety and C6-C12 aryl;
x represents the number of coordinating groups in the organic ligand, and is 1, 2 or 3, preferably 1 or 2;
n represents a coordination number, and is a positive number from 1 to 6, preferably from 2 to 5; and
y represents the number of oxygen atom linked to the metal M₁ via a non-coordination bond, and is a positive number from 0 to 3, preferably from 1 to 3.

4. The unsupported hydrogenation catalyst according to claim 3, wherein, in the infrared spectrum of the catalyst, the distance between a characteristic peak at the position of 1350-1450 cm⁻¹ and a characteristic peak at the position of 1500-1610 cm⁻¹ is less than 145 cm⁻¹.

5. The unsupported hydrogenation catalyst according to claim 1, wherein at least a portion of the complex in the catalyst has a structure represented by formula (I-2): 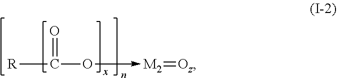
wherein M₂ represents a metal, and is at least two selected from the group consisting of Group VB metals, Group VIB metals, Group VIII metals, and Group IB metals that have a hydrogenation activity;
→ represents a coordination bond;
R represents a C3-C19 hydrocarbyl group, and is preferably selected from the group consisting of C5-C11 normal alkyl, C5-C11 isomeric alkyl, C5-C12 alkyl with cycloalkyl moiety and C6-C12 aryl;
x represents the number of coordinating groups in the organic ligand, and is 1 or 2, preferably 1;
n represents a coordination number, and is a positive number from 1 to 6, preferably from 2 to 5;
z represents the number of oxygen atom linked to the metal M₂ via a non-coordination bond, and is a positive number from 0 to 3, preferably from 1 to 3.

6. The unsupported hydrogenation catalyst according to claim 5, wherein, in the infrared spectrum of the catalyst, the distance between a characteristic peak at the position of 1350-1450 cm⁻¹ and a characteristic peak at the position of 1500-1610 cm⁻¹ is more than 145 cm⁻¹.

7. The unsupported hydrogenation catalyst according to claim 1, wherein the Group VB metals, Group VIB metals, Group VIII metals, and Group IB metals having a hydrogenation activity are selected from the group consisting of V, Cr, Mo, W, Fe, Co, Ru, Ni, Cu, and Pd, preferably selected from the group consisting of Mo, Ni, W, Fe, V, and Co.

8. The unsupported hydrogenation catalyst according to claim 1, wherein the organic ligand is derived from a C4-C20 organic carboxylic acid, preferably from one or more selected from the group consisting of C4-C20 normal or isomeric alkyl carboxylic acids, C6-C20 naphthenic carboxylic acids comprising a saturated carbon ring and C7-C20 aromatic carboxylic acids comprising an aromatic ring, more preferably from one or more selected from the group consisting of C6-C12 normal or isomeric alkyl carboxylic acids, C6-C13 naphthenic carboxylic acids comprising a saturated carbon ring and C7-C13 aromatic carboxylic acids comprising an aromatic ring, further preferably from one or more selected from the group consisting of succinic acid, hexanoic acid, adipic acid, heptanoic acid, octanoic acid, nonanoic acid, ethylhexanoic acid, oleic acid, petroleum acid, salicylic acid, benzoic acid and phenylacetic acid.

9. The unsupported hydrogenation catalyst according to claim 1, wherein the catalyst has a metal content of from 5% to 35%, preferably from 8% to 30%, more preferably from 10% to 25%, particularly preferably from 10% to 20%, calculated based on metal and relative to the weight of the catalyst.

10. The unsupported hydrogenation catalyst according to claim 1, which is obtained by directly reacting an elemental metal selected from Group VB metals, Group VIB metals, Group VIII metals, Group IB metals or combinations thereof that have a hydrogenation activity, its oxide, its hydroxide, its metallic oxyacid and/or its metal inorganic salt with an organic ligand compound selected from C4-C20 organic carboxylic acids, preferably selected from the group consisting of C4-C20 normal or isomeric alkyl carboxylic acids, C6-C20 naphthenic carboxylic acids comprising a saturated carbon ring, C7-C20 aromatic carboxylic acids comprising an aromatic ring or combinations thereof, more preferably selected from the group consisting of C6-C12 normal or isomeric alkyl carboxylic acids, C6-C13 naphthenic carboxylic acids comprising a saturated carbon ring, C7-C13 aromatic carboxylic acids comprising an aromatic ring or combinations thereof, further preferably selected from the group consisting of succinic acid, hexanoic acid, adipic acid, heptanoic acid, octanoic acid, nonanoic acid, ethylhexanoic acid, oleic acid, petroleum acid, salicylic acid, benzoic acid, phenylacetic acid or combinations thereof.

11. A method for preparing the unsupported hydrogenation catalyst according to claim 1, comprising the steps of:
mixing a metal source or a dispersion thereof with an organic ligand compound;
reacting the mixture obtained in step 1) for 1-8 h at 100-350° C.; and
collecting the resulting liquid product,
wherein the metal source is selected from the group consisting of elemental metal, metal oxide, metal hydroxide, metallic oxyacid, metal inorganic salt or combinations thereof, and the metal in the metal source is selected from the group consisting of Group VB metals, Group VIB metals, Group VIII metals, Group IB metals, and combinations thereof that have a hydrogenation activity;
the organic ligand compound is selected from the group consisting of C4-C20 organic carboxylic acids or anhydrides thereof, preferably selected from the group consisting of C4-C20 normal or isomeric alkyl carboxylic acids, C6-C20 naphthenic carboxylic acids comprising a saturated carbon ring, C7-C20 aromatic carboxylic acids comprising an aromatic ring, anhydrides thereof or combinations thereof; and
the molar ratio of the organic ligand compound to the metal in the metal source is 1-10:1.

12. The method according to claim 11, wherein the mixture obtained in step 1) is consisted of the metal source and the organic ligand compound; or
the mixture obtained in step 1) is consisted of the metal source, a dispersion medium for dispersing the metal source and the organic ligand compound.

13. The method according to claim 11, wherein in step 1), a dispersion of the metal source is used, the dispersion medium in the dispersion is an inorganic dispersion medium selected from water, carbonic acid, hydrochloric acid, sulfuric acid or phosphoric acid or an organic dispersion medium selected from the group consisting of ethanol, toluene, xylene, petroleum ether, gasoline, diesel oil, or combinations thereof;
preferably, the weight ratio of the dispersing medium to the metal source in the dispersion is 1-25:1, more preferably 2-8:1.

14. The method according to claim 11, wherein in step 2), a reaction temperature of 160-260° C. and the reaction time is 2-5 h.

15. A hydrogenation catalyst composition, comprising the unsupported hydrogenation catalyst according to claim 1 and at least one organic ligand compound and/or at least one organic solvent, wherein:
the organic ligand compound is selected from C4-C20 organic carboxylic acids, preferably selected from the group consisting of C4-C20 normal or isomeric alkyl carboxylic acids, C6-C20 naphthenic carboxylic acids comprising a saturated carbon ring, C7-C20 aromatic carboxylic acids comprising an aromatic ring or combinations thereof, more preferably selected from the group consisting of C6-C12 normal or isomeric alkyl carboxylic acids, C6-C13 naphthenic carboxylic acids comprising a saturated carbon ring, C7-C13 aromatic carboxylic acids comprising an aromatic ring or combinations thereof, further preferably selected from the group consisting of succinic acid, hexanoic acid, adipic acid, heptanoic acid, octanoic acid, nonanoic acid, ethylhexanoic acid, oleic acid, petroleum acid, salicylic acid, benzoic acid, phenylacetic acid or combinations thereof;
the organic solvent is selected from the group consisting of aliphatic hydrocarbons, aromatic hydrocarbons, alicyclic hydrocarbons, halogenated hydrocarbons, alcohol solvents, ether solvents, ester solvents, ketone solvents or combinations thereof, preferably selected from the group consisting of toluene, gasoline, ethanol, diesel oil or combinations thereof.

16. The composition according to claim 15, wherein the composition comprises at least one of the organic ligand compounds and the composition shows an infrared spectrum with characteristic peaks at positions of 700-1000 cm⁻¹, 1350-1450 cm⁻¹, 1500-1610 cm⁻¹ and 1700-1750 cm⁻¹.

17. The composition according to claim 15, wherein the unsupported hydrogenation catalyst is present in an amount of from 50% to 95%, preferably from 80% to 95%; and the total amount of the organic ligand compound and the organic solvent is from 5% to 50%, preferably from 5% to 20%, based on the weight of the composition.

18. Use of the unsupported hydrogenation catalyst according to claim 1 in the hydrogenation of hydrocarbons, wherein the hydrocarbonaceous feedstock is an unsaturated hydrocarbon compound, such as benzene, alkylbenzene, naphthalene, alkylnaphthalene, anthracene, alkylanthracene, and the like; or a mixture comprising unsaturated hydrocarbon compounds such as crude oil, gasoline, diesel oil, vacuum gas oil, residual oil, and the like.

19. An unsupported catalyst composition suitable for the hydrogenation of heavy oils, comprising, by weight, from 10% to 45% of a hydrogenation catalyst component, from 45% to 80% of a dispersing medium and from 1.0% to 10% of an activator, wherein:
the hydrogenation catalyst component is consisted of the unsupported hydrogenation catalyst according to claim 1 and optionally an organic ligand compound selected from C4-C20 organic carboxylic acids,
the dispersion medium is selected from the group consisting of organic solvents, petroleum fractions or combinations thereof, the organic solvent is selected from the group consisting of aliphatic hydrocarbons, aromatic hydrocarbons, alicyclic hydrocarbons, halogenated hydrocarbons, alcohol solvents, ether solvents, ester solvents, ketone solvents or combinations thereof, the petroleum fraction is selected from distillate oils with a distillation range of 150-524° C. or residual oil components with a boiling point >524° C.,
the activator is selected from the group consisting of elemental sulphur, sulphur-containing compounds, or combinations thereof, preferably selected from the group consisting of thiols, thioethers, carbon disulphide, sulphur, thiophenic compounds, or combinations thereof.

20. The unsupported catalyst composition according to claim 19, wherein the organic ligand compound is selected from the group consisting of C4-C20 normal or isomeric alkyl carboxylic acids, C6-C20 naphthenic carboxylic acids comprising a saturated carbon ring, C7-C20 aromatic carboxylic acids comprising an aromatic ring, or combinations thereof, preferably selected from the group consisting of C6-C12 normal or isomeric alkyl carboxylic acids, C6-C13 naphthenic carboxylic acids comprising a saturated carbon ring, C7-C13 aromatic carboxylic acids comprising an aromatic ring, or combinations thereof, further preferably selected from the group consisting of succinic acid, hexanoic acid, adipic acid, heptanoic acid, octanoic acid, nonanoic acid, ethylhexanoic acid, oleic acid, petroleum acid, salicylic acid, benzoic acid, phenylacetic acid, or combinations thereof.

21. The unsupported catalyst composition according to claim 19, wherein the hydrogenation catalyst component has a metal content of from 5% to 35%, preferably from 8% to 30%, more preferably from 10% to 25%, particularly preferably from 10% to 20%, and an organic ligand compound content of from 0% to 50%, preferably from 5% to 50%, more preferably from 5% to 20%, based on the weight of the hydrogenation catalyst component.

22. (canceled)

23. Use of the unsupported catalyst composition according to claim 19 in the hydro-upgrading of heavy oils.

24. A process for the hydro-upgrading of heavy oils, comprising the step of subjecting a heavy oil feedstock to a hydro-upgrading reaction under heating conditions in the presence of hydrogen and the unsupported catalyst composition according to claim 19, which is optionally presulphurized.

25. The use according to claim 23, wherein the conditions of the hydro-upgrading include: an amount of the unsupported catalyst composition of 50-10000 μg/g, calculated based on metal and relative to the weight of the heavy oil feedstock, an initial hydrogen pressure of 5-20 MPa, a reaction temperature of 360-480° C., a liquid hourly space velocity of 0.05-2.0 h⁻¹, and a hydrogen-to-oil volume ratio of 300-2000;
preferably, the conditions of the hydro-upgrading include: an amount of the unsupported catalyst composition of 50-3000 μg/g, calculated based on metal and relative to the weight of the heavy oil feedstock, an initial hydrogen pressure of 5-15 MPa, a reaction temperature of 390-450° C., a liquid hourly space velocity of 0.05-1.0 h⁻¹, and a hydrogen-to-oil volume ratio of 500-1500.
